(19) United States
(12) Patent Application Publication
(10) Pub. No.: US 2013/0256132 A1
(43) Pub. Date: Oct. 3, 2013

(54) Test strip structure

(71) Applicant: Erik Chen, New Taipei City (TW)

(72) Inventor: Erik Chen, New Taipei City (TW)

(21) Appl. No.: 13/434,872

(22) Filed: Mar. 30, 2012

(51) Int. Cl. G01N 27/327; G01N 33/66

(52) U.S. Cl. 204/403.02

(57) ABSTRACT

A test strip structure is provided, and more particularly to a blood glucose test strip, which facilitates permeating blood, comprises a plurality of conductive strips disposed over a top of a base layer, and a first insulation glue layer covering with the conductive strip. A roof plate layer is disposed over a top of the first insulation glue layer. One end of the conductive strip is capable of receiving a detection of a blood glucose machine while another end is disposed with a reaction layer. The first insulation glue layer is formed with a notch portion relative to the reaction layer. The notch portion is formed with a notch port, which is at one end of the base layer, capable of providing blood to permeate into the reaction layer. The first insulation glue layer has a ventilation gutter communicating with the notch portion.

### BACKGROUND OF THE INVENTION

1. Field of the Invention

The present invention relates to a test strip structure, and more particularly to a blood glucose test strip that facilitates permeating blood and relates to a substratum, a conductive strip, a reaction layer, a first insulation glue layer and a roof plate layer, especially for a notch portion, a notch port, a ventilation gutter and a ventilation port of the first glue layer.

2. Description of the Related Art

Diabetes mellitus is one of diseases of civilization. When insulin secretion within blood is not enough, blood glucose may not be effectively converted into energy. Surplus blood glucose will be accumulated in the body to cause apoplexy or other diseases. A patient must be confirmed whether or not food and drink and exercise are normal by intensively measuring blood glucose even if he/she is in a process of taking medicine, thereby preventing sickness.

A conventional manner of detecting blood glucose mainly utilizes a glucose test strip to detect blood. The glucose test strip is an advanced glucose sensor and needs to incorporate with a glucose meter to measure glucose content within blood through electrochemical analysis. The conventional glucose test strip has several conductive strips. Two ends of each conductive strip are respectively formed with an electrode and a detection terminal. The electrode is covered with a reaction layer having glucose oxidase. When blood permeates into the reaction layer, electrochemical reaction occurs to generate an electron signal. The electron signal is delivered to the detection terminal through the conductive strip. Afterward the glucose test strip is inserted into the access notch of the glucose meter to read the glucose content within blood.

In addition, to easily permeate blood into the reaction layer, the conventional glucose test strip is disposed with a plurality of ventilation pores communicating the reaction layer. However, merely disposing the ventilation pore on the glucose test strip is difficult to improve the speed of permeating blood into the reaction layer. Further, blood may slowly permeate into the reaction layer.

The inventor has tried to increase the pore diameter of the ventilation pore to enhance the speed of permeating blood into the reaction layer. However, the contact area between the reaction layer and air is also increased, and the reaction layer is easily affected by air to cause oxidization that needs to be overcome.

### SUMMARY OF THE INVENTION

In view of the shortcomings of the prior art, the inventor(s) of the present invention based on years of experience in the related industry to conduct extensive researches and experiments, and finally developed a blood glucose test strip as a principle objective to facilitate permeating blood to overcome the problem of easily slowing down the blood permeation since the ventilation pore is merely disposed on a conventional blood glucose test strip such that the smoothness of permeating blood can be improved.

To achieve the foregoing object, the test strip structure of the invention comprises a plurality of conductive strips distributed on a top of a base layer, and a first insulation glue layer covering the conductive strip, and the a top of the first insulation glue layer disposed with a roof plate layer, one end of the conductive strip receiving a detection of a blood glucose machine while another end of the conductive strip disposed with a reaction layer, the first insulation glue layer formed with a notch portion relative to the reaction layer, the notch portion formed with a notch port, which is at one end of the base layer, capable of providing blood to permeate into the reaction layer, wherein the first insulation layer has a ventilation gutter communicating with the notch portion, and the ventilation gutter is formed with a ventilation port at another side of the base layer. The ventilation gutter and the ventilation port are capable of increasing a speed of blood that permeates into reaction layer from the notch port.

With the foregoing features, the notch portion communicates atmosphere through the notch port, the ventilation gutter and the ventilation port. When blood is in contact with the notch port, blood can permeate into the reaction layer in the notch portion through the notch port, and air inside the notch portion can communicate with atmosphere through the ventilation gutter and the ventilation port.

Accordingly, under a premise of no increasing the contact area between the reaction layer and air, blood can be facilitated permeating into the reaction layer to further improve the smoothness of blood permeation, and the reaction layer can be prevented from oxidized due to air influence.

The following is further depicted with preferred embodiments.

In a preferred embodiment, double sides of the conductive strip are respectively formed with a detection terminal tested by the blood glucose machine, and an electrode, and the reaction layer is covered over the electrode.

To easily attach blood to the notch port, the roof plate layer is formed with an arc gap relative to the notch port so that blood can be attached to the gap and permeates into the notch port along the gap.

To further improve the smoothness of permeating blood, the roof plate layer is formed with a ventilation seam relative to the reaction layer so that air in the notch portion communicates with atmosphere through the ventilation seam.

Alternatively, the ventilation seam is also used to be in contact with blood to allow blood permeating into the reaction layer through the ventilation seam, and air inside the notch portion communicates with atmosphere through the notch port, the ventilation gutter and the ventilation port.

The notch port and the ventilation port are respectively at two adjacent end edges of the same included angle of the base layer.

A second insulation glue layer is disposed between the first insulation glue layer and the base layer, wherein the second insulation glue layer is formed with a frame body relative to the notch portion to contain the reaction layer to allow the notch portion communicating with the frame notch.

The second insulation glue layer is formed with a gutter portion relative to the ventilation gutter, and an insulation point spaced between the frame notch and the gutter portion, and the gutter portion communicates with the ventilation gutter. The reaction layer is restricted by the insulation point to form in the frame notchm, and a height of the reaction layer equals a height of the frame notch such that a bottom of the roof plate layer is prevented from being touched with the reaction layer to cause an obstruction between the notch port and the ventilation port, and ventilation is achieved among the notch port, the notch portion, the ventilation gutter and the ventilation port to avoid a situation of blood that does not easily permeate into the reaction layer The notch port is at two sides or one side of the test strip.

### BRIEF DESCRIPTION OF THE DRAWINGS

FIG. 1 is a three-dimensional drawing according to a preferred embodiment the invention;

FIG. 2 is a plane diagram according to FIG. 1;

FIG. 3 is a three-dimensional decomposition drawing according to FIG. 1;

FIG. 4 is a partial enlarged drawing according to an A-A section shown in FIG. 2;

FIG. 5 is a cross-sectional drawing of an added implementation type according to FIG. 4;

FIG. 6 is a three-dimensional drawing of using status according to the embodiment shown in FIG. 1.

### DETAILED DESCRIPTION OF THE PREFERRED EMBODIMENTS

The foregoing and other technical characteristics of the present invention will become apparent with the detailed description of the preferred embodiments and the illustration of the related drawings.

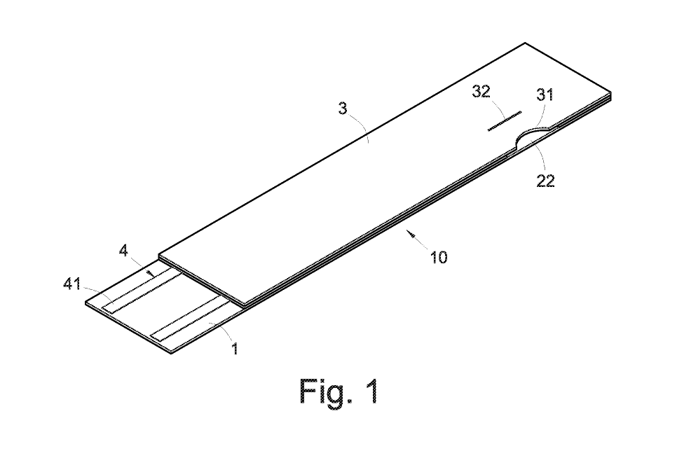

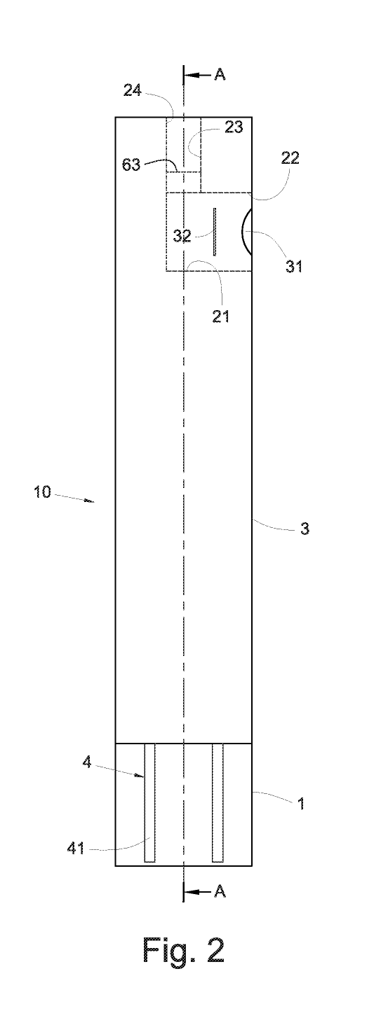

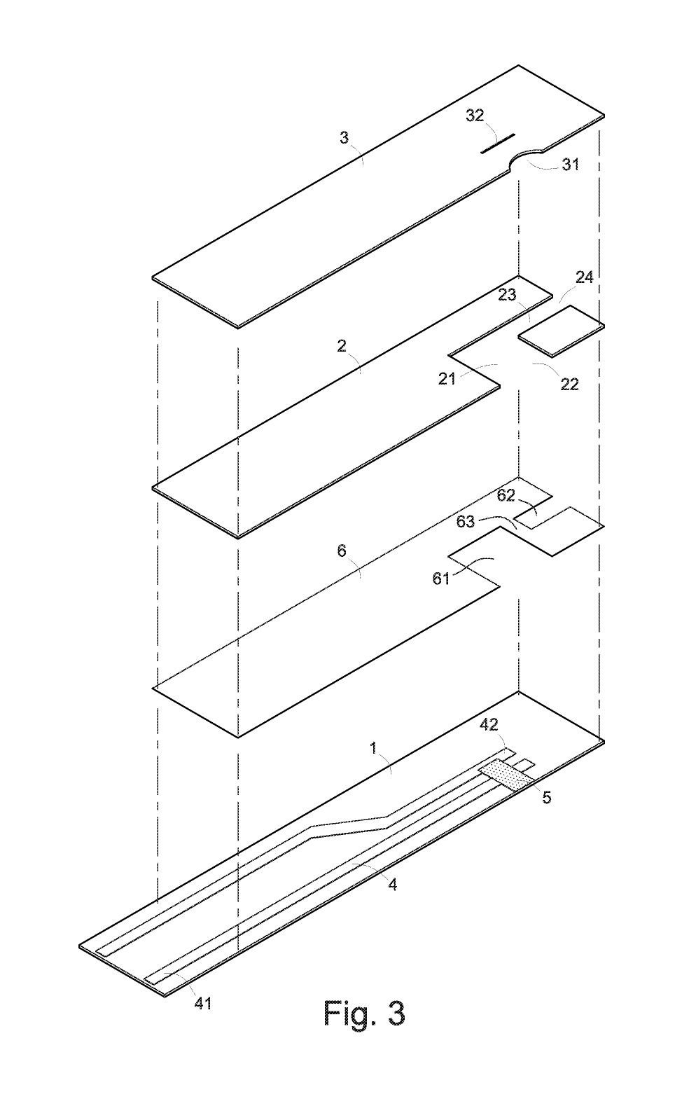

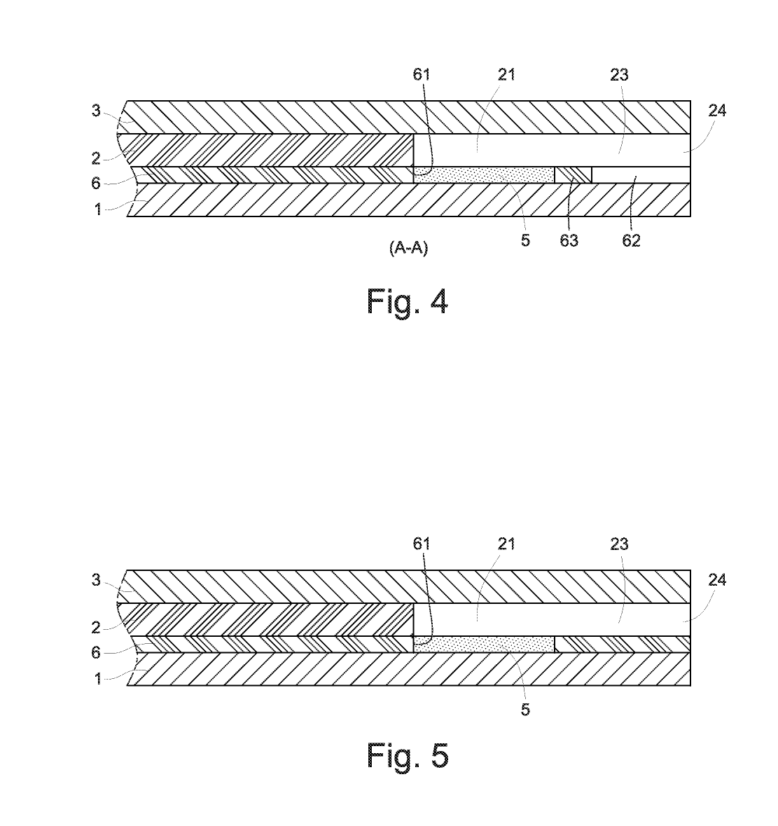

With reference to FIG. 1 for a three-dimensional drawing according to a preferred embodiment of the invention is depicted, and with reference to FIG. 2 to FIG. 4 for a test strip structure of the invention are depicted. The test strip 10 includes abase layer 1, a first glue layer 2 for insulation and a roof plate layer 3 for protection, wherein the base layer 1 is a bar shape, and a top of the base layer 1 is distributed with a plurality of conductive strips 4. The first insulation glue layer 2 is disposed at the top of the base layer 2.

One end of the conductive layer 4 can receive a detection of an external blood glucose machine 7. Another end of the conductive strip 4 is disposed with a reaction layer 5. The first insulation glue layer 2 is formed with a notch portion 21 related to the reaction layer 5.

The notch portion 21 is formed with a notch port 22, which is at one end of the base layer 1, capable of providing blood to permeate into the reaction layer 5, wherein the first insulation glue layer 2 has a ventilation gutter 23 communicating with the notch portion 21. The ventilation gutter 23 is formed with a ventilation port 24 at another side of the base layer 1. The ventilation gutter 23 and the ventilation port 24 are capable of increasing a speed of blood that permeates into reaction layer 5 from the notch port 22

In a preferred embodiment, two ends of the conductive strip are extended toward two ends of the base layer 1 to respectively form a detection terminal 41 capable of being detected by the blood glucose machine 7 and an electrode 42.

The reaction layer 5 is covered with the electrode 42, and the reaction layer 5 can include glucose oxidase.

The detection terminal 41 can be inserted into an access notch 71 of the blood glucose machine 7 in accordance with the base layer 1 so as to be detected by the blood glucose machine 7.

The electrode 42 can be a dissolved oxygen electrode, a platinum electrode, an ammonia electrode, a carbon dioxide electrode or a PH electrode.

The reaction layer 5 can be permeated by blood of a tester to generate an electron signal. Manners of outputting the electron signal can include current, voltage and electro conductivity measurements. The electrode 42 and the reaction layer 5 can be formed into an enzyme electrode, wherein its measurement principle uses the specific electrode to measure the production of reacted products or extinction of reactants. The blood glucose machine 7 can sense the electron signal through the detection terminals 41 and measures the concentration of glucose within blood through current measurement.

The roof plate layer 3 is formed with an arc gap 31 relative to the notch port 22 to allow blood to attach the gap 31 so that blood can easily attach the notch port 22 and permeate into the reaction layer 5 of the notch portion 21 along the gap 31 and the notch port 22.

The roof plate layer 3 is further formed with a ventilation seam 32 relative to the reaction layer 5 to allow air within the notch portion 21 communicating with atmosphere through the ventilation seam 32, thereby further improving the smoothness for blood permeating into the reaction layer.

The notch port 22 and the ventilation port 24 are respectively at two adjacent end edges of the same included angle of the base layer 1.

A second insulation glue layer 6 is disposed between the first insulation glue layer 2 and the base layer 1. The second insulation glue layer 6 is formed with a frame notch 61, which contains the reaction layer 5, relative to the notch portion 21 to allow the notch portion 21 communicating with the frame notch 61. The height of the reaction layer 5 is equivalent to the height of the frame notch 61, and the frame notch 61 and the notch port 33 communicate to each other.

The second insulation glue layer 6 is formed with a gutter portion 62 relative to the ventilation gutter 23 and has an insulation point 63 spaced between the frame notch 61 and the gutter portion 62. The gutter portion 52, the ventilation gutter 23 and the ventilation port 24 communicate to each other.

The notch port 22 can be located at two sides of the test strip 10 in the embodiment or can be located at one side of the test strip 10.

With the foregoing structures, the invention can be implemented. With the design of the insulation point 63, the reaction layer 5 is formed in the frame notch 61 through the restriction of the insulation point 63, and the height of the reaction layer 5 is equivalent to the height of the frame notch 61 such that the reaction layer 5 can be prevented from touching a bottom of the roof plate layer 3 to cause an obstruction between the notch port 22 and the ventilation port 24, resulting in ventilation among the notch port 22, the notch portion 21, the ventilation gutter 23 and the ventilation port 24. The situation for blood that does not easily permeate into the reaction layer 5 can be avoided, and the ventilation of the ventilation gutter 23 can be increased by the design of the gutter portion 62.

While using the blood test strip 10 of the invention, a blood sample is placed at a side of the gap 31. Since the seam type notch port 22 having tiny distance is between the roof plate layer 3 and the base layer 1, the blood sample is absorbed to the reaction layer 5 through the capillary effect generated by the seam type notch port 22 and the touch portion 21.

The ventilation seam 32, the ventilation gutter 23, the gutter portion 62 and the ventilation port 24 can increase the speed of absorbing the blood sample. When the blood sample is fully distributed the reaction layer 5, the reaction layer 5 reacts with the blood sample to generate the electron signal.

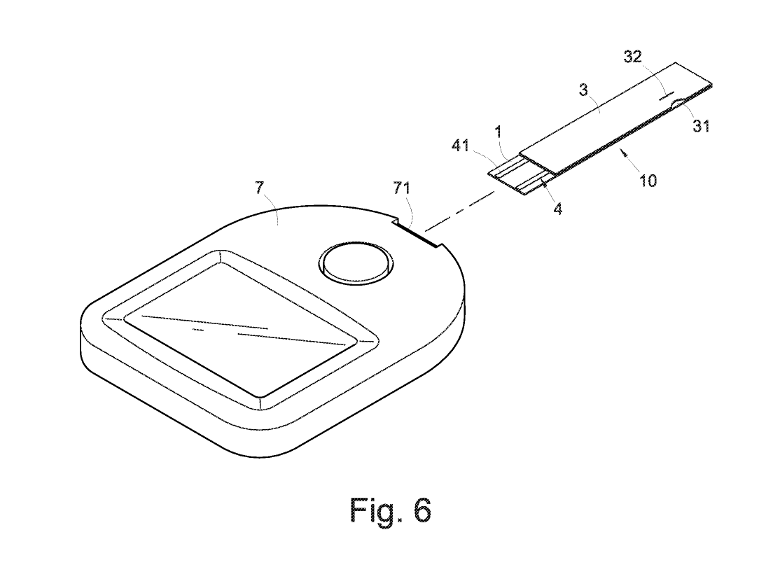

Next, the detection terminal 41 of the blood glucose test strip 10 is inserted into the access notch 71 (as shown in FIG. 6) of the blood glucose machine 7 to allow the blood glucose machine 7 to read the electron signal through the detection terminal 41 so as to measure the concentration of glucose within blood.

The notch portion 21 can communicate atmosphere through the notch port 22, the ventilation gutter 23, the gutter portion 62, the ventilation port 24 and the ventilation seam 32. When blood is in contact with the notch port 22, blood can permeate into the reaction layer 5 in the notch portion 21 through the notch port 22, and air within the notch portion 21 can communicate with atmosphere by passing through the ventilation gutter 23, the gutter portion 62, the ventilation port 24 and the ventilation seam 32.

Accordingly, under a premise of no increasing the contact area between the reaction layer 5 and air, with the design of the ventilation gutter 23, the gutter portion 62, the ventilation port 24 and the ventilation seam 32, blood can be speeded up to permeate into the reaction layer 5 to overcome the problem of easily slowing down the blood permeation since the ventilation pore is merely disposed on a conventional blood glucose test strip such that the smoothness of permeating blood can be improved, and the reaction layer 5 can be prevented from being affected by air to cause oxidation.

It should be noted that when the ventilation gutter 23, the gutter portion 62 and the ventilation port 24 are carelessly blocked, air within the notch portion 21 can communicate with atmosphere through the ventilation seam 32.

Of course, the ventilation seam 32 can also be used to be in contact with blood to permeate blood into the reaction layer 5 through the ventilation seam 32, and air within the notch portion 21 can communicate with atmosphere by simultaneously passing through the notch port 22, the ventilation gutter 23 and the ventilation port 24 to improve the smoothness of permeating blood.

In addition, the gutter portion 62 and the insulation point 63 on the second insulation glue layer 6 can be omitted (as shown in FIG. 5). Air within the notch portion 21 can communicate with atmosphere through the ventilation gutter 23 and the ventilation port 24.

The invention improves over the prior art and complies with patent application requirements, and thus is duly filed for patent application. While the invention has been described by device of specific embodiments, numerous modifications and variations could be made thereto by those generally skilled in the art without departing from the scope and spirit of the invention set forth in the claims.

What is claimed is:

1. A test strip structure comprising:
a plurality of conductive strips distributed on a top of a base layer, and a first insulation glue layer covering the conductive strip, and the a top of the first insulation glue layer disposed with a roof plate layer, one end of the conductive strip receiving a detection of a blood glucose machine while another end of the conductive strip disposed with a reaction layer, the first insulation glue layer formed with a notch portion relative to the reaction layer, the notch portion formed with a notch port, which is at one end of the base layer, capable of providing blood to permeate into the reaction layer, the characterized by that the first insulation glue layer having a ventilation gutter communicating with the notch portion, the ventilation gutter formed with a ventilation port at another side of the base layer, the ventilation gutter and the ventilation port capable of increasing a speed of blood that permeates into reaction layer from the notch port;
wherein the test strip structure further comprising a second insulation glue layer disposed between the first insulation glue layer and the base layer, wherein the second insulation glue layer is formed with a frame body relative to the notch portion to contain the reaction layer to allow the notch portion communicating with the frame notch and the second insulation glue layer is formed with a gutter portion relative to the ventilation gutter, and an insulation point spaced between the frame notch and the gutter portion, and the gutter portion communicates with the ventilation gutter, and the reaction layer is restricted by the insulation point to form in the frame notch, and a height of the reaction layer equals a height of the frame notch such that a bottom of the roof plate layer is prevented from being touched with the reaction layer to cause an obstruction between the notch port and the ventilation port, and ventilation is achieved among the notch port, the notch portion, the ventilation gutter and the ventilation port to avoid a situation of blood that does not easily permeate into the reaction layer.

2. The test strip structure as recited in claim 1, wherein double sides of the conductive strip are respectively formed with a detection terminal tested by the blood glucose machine, and an electrode, and the reaction layer is covered over the electrode.

3. The test strip structure as recited in claim 1, wherein the roof plate layer is formed with an arc gap relative to the notch port.

4. The test strip structure as recited in claim 1, wherein the roof plate layer is formed with a ventilation seam relative to the reaction layer.

5. The test strip structure as recited in claim 1, wherein the notch port and the ventilation port are respectively at two adjacent end edges of the same included angle of the base layer.

6. (canceled)

7. The test strip structure as recited in claim 1, wherein the notch port is at two sides or one side of the test strip.
